(19) United States
(12) Patent Application Publication
(10) Pub. No.: US 2011/0091926 A1
(43) Pub. Date: Apr. 21, 2011

(54) PERFUSABLE BIOREACTOR FOR THE PRODUCTION OF HUMAN OR ANIMAL TISSUES

(75) Inventor: Bernhard Frerich, Rostock (DE)

(73) Assignee: NOVATISSUE GMBH, Leipzig (DE)

(21) Appl. No.: 12/934,491

(22) PCT Filed: Mar. 23, 2009
(86) PCT No.: PCT/EP09/02109

§ 371 Date: Dec. 20, 2010

(30) Foreign Application Priority Data
Mar. 25, 2008 (DE) 10 2008 015 634.5
Mar. 25, 2008 (DE) 10 2008 015 635.3

(51) Int. Cl. C12Q 1/02; C12M 3/00; C12N 5/00

(52) U.S. Cl. 435/29; 435/289.1; 435/395; 435/373; 435/400; 435/402

(57) ABSTRACT

A perfusable bioreactor is used for the production of human tissues, animal tissues or tissue equivalents. Their production is based on a structure cultivated in the interior, the interior being enclosed by an envelope and containing at least one inlet and one outlet for a liquid nutrient medium. Accordingly, the bioreactor can be connected to a unit for producing a perfusion pressure of the nutrient medium. This tissue replacement is particularly suitable for use in clinical/therapeutical applications.

The invention relates to a perfusable bioreactor for producing human or animal tissues or tissue equivalents, wherein the production of these is based on a structure cultivated in the internal space, the internal space is enclosed by an envelope and comprises at least one inlet and one outlet for a liquid nutrient medium, the bioreactor being connectable to a unit for generating perfusion pressure of the nutrient medium. This tissue replacement is used, in particular, for clinical-therapeutic applications.

Within the scope of the invention, “perfusable bioreactors” are bioreactors that allow a liquid medium primarily to flow through the structure introduced therein and secondly to flow around it.

Within the scope of the invention, “structures” are artificially produced three-dimensional tissue equivalents containing living cells in a three-dimensional matrix, more particularly combinations of scaffolds and living cells (scaffold-cell combinations), possibly also combined with matrix factors.

Within the scope of the invention, the terms blood-vessel equivalents and blood-vessel-wall equivalents are used analogously to this definition.

Many different types of perfusable bioreactors have been developed to date for in vitro production of tissues. However, the focus up until now has mainly been on producing bioreactors with rigid walls, the shape of which has not been matched to the tissue to be cultivated. Hence loads and influences on the tissue growing in vitro do not correspond to those on a natural tissue in vivo. However, the tissue growth in vivo is precisely influenced to an extent that should not be underestimated by mechanical loads and these likewise should be modeled in vitro. Moreover, it is disadvantageous in that even if perfusion through the tissue or tissue equivalent is intended, it is generally not perfusion in the actual sense of the word, but rather a flow around it, and hence the interior of such a structure is not supplied in an optimum fashion.

This problem particularly plays a role in soft tissue and blood vessels. The provision or production of food by and perfusion through blood vessels is a problem that in essence is yet to be solved in tissue engineering (cell and tissue cultivation). It is already important even in small tissue volumes for a vessel system or a corresponding equivalent to be implemented because diffusion no longer suffices for feeding at distances of more than approximately 100-300 μm to the next blood capillary. Hence, such a tissue also requires its own blood-vessel supply, which, naturally, must be matched to the shape of the implant. Thus, there is a need for bioreactors in which supplying blood vessels can be cultivated in conjunction with any tissue and that moreover satisfy the physical/mechanical requirements of soft-tissue and/or vascular/microvascular engineering.

Although previous bioreactors have already implemented pulsating perfusions, which are intended to simulate blood pressure, in particular to condition artificial vessel structures to the blood pressure forces in vivo, said pulsating perfusions often are not physiological or are reflected in a non-physiological fashion in the surroundings of a rigid bioreactor wall, which can also lead to the destruction of the cells in the reactor. Thus, the provision of physiological tissue compliance (elasticity of the tissue) in the three-dimensional environment is required, and previous systems do not allow this.

A further problem lies in the design of the shape of tissue structures, which should compensate for e.g. defects in the subcutaneous fat tissue in the vicinity of the surface.

The tissue to be produced requires an individual shape, i.e. a defined spatial configuration, depending on the respective use, in particular as a tissue replacement.

Herein, it is particularly important for the tissue structure to fill the defect as precisely as possible after the implantation in order to obtain the desired aesthetic result. It is desirable for a specific shape to be obtained in a targeted fashion for soft-tissue engineering, more particularly for fat-tissue engineering for shaping the surface or for compensating for a defect, but also for a bone that is used in contour-effective localizations.

It is also important with respect to establishing the blood supply for the shape already to be taken into account when the tissue is being produced so that the blood-vessel supply is immediately set up in a dimensionally accurate fashion and is not subsequently destroyed by corrections in the shape.

In previous approaches, the external shape of a tissue structure produced by means of tissue engineering was generally attempted to be pre-shaped and set using the shape of a scaffold on which the cells grow and reproduce. The external shape of the scaffold forms the guide rail in which the artificial tissue forms with specific differentiation.

However, an ideal approach would allow work without a scaffold or with quickly resorbable scaffold materials. However, in that case, the external shape must be prescribed at the same time and, ideally, reproduce the individual defect into which the structure should later fit.

Even when using scaffolds, the cultivation in perfusion bioreactors, which have not been matched to the scaffold in terms of their shape, harbors a possible disadvantage. The nutrient medium often only circulates in these bioreactors rather than perfusing, and so the central scaffold components may not receive enough food.

An object of the invention is to provide a bioreactor that overcomes the disadvantages of the prior art.

The object of the invention is achieved by the features of claim 1.

What is essential to the invention is that at least one subsegment of this envelope consists of an elastic material.

It is essential that the envelope of the bioreactor is elastic in large parts and the elasticity thereof together with that of the tissue or vessel equivalent in the interior has a mechanical “compliance” corresponding to that of the target tissue.

The elastic subsegments of the envelope ensure that physiological mechanical loads (pressures and forces) are exerted, e.g. by a pulsating perfusion from the inside at pressures that are specifically in the physiological or pathological range (blood pressure).

Thus, by way of example, a pulsatile perfusion is transmitted against the elastic envelope (wall) by the tissue and the hydrostatic pressure of the nutrient medium, and mechanical or hydrodynamic loads can act on the tissue. The essential difference to previous solutions lies in the fact that these perfusion dynamics are brought about in elastic surroundings and the compliance of natural blood vessels and tissues in physiological and pathological situations can be modeled by adjusting the elasticity of the chamber wall.

This can also be in conjunction with a tight-fitting, interlocking design of the reactor wall with respect to the tissue to be produced, which is advantageous in that the tissue stretch can be set by the elasticity of the reactor wall. A further advantage lies in the fact that the dimensionally accurate enveloping supports perfusion through the scaffold and simple circulation of the medium is avoided. The contour of the internal space of the bioreactor then substantially corresponds to the external contour of the tissue to be produced.

It moreover is preferable for the internal contour of the internal space to correspond to the external contour of the human or animal tissue or tissue equivalent to be produced over at least more than 50% of the surface.

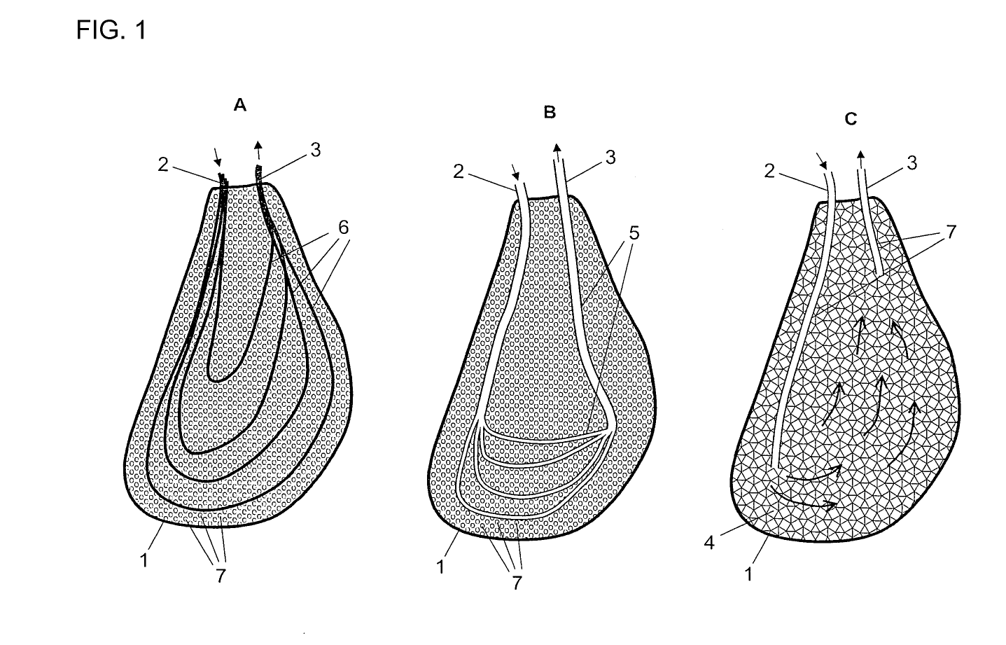

In contrast to other bioreactors, the structure largely fills the bioreactor according to the invention and the supply is not brought about by the medium circulating the structure, but primarily by perfusion through a hollow fiber or hollow line system, a pre-shaped or growing artificial vessel system, a porous scaffold or a combination of at least two of these principles (illustrated in FIGS. 1A to 1C).

The solution according to the invention thus also contains the individual, dimensionally accurate enveloping of a scaffold 4 in an individual shape such that the scaffold is tightly surrounded by the elastic envelope 1, i.e. the elastic chamber wall, and perfused by the perfusion medium.

The perfusion, the regulation of pressures and forces and the tissue compliance at physiological boundary values should allow developing of a microvascular vessel network that will ultimately take over the supply. Said microvascular vessel network will obtain its central inlet 2 and outlet 3 from the pre-shaped channels or resorbable hollow fiber tubes, and so these connections can be connected by microsurgical means to established blood vessels in the receiver camp as artificial supplying blood vessels, and hence perfusion of the tissue is ensured directly after the implantation.

The pre-shaping, i.e. the defined spatial configuration, of the tissue to be implanted (tissue implant), which has the spatial configuration of the tissue produced with the bioreactor according to the invention, is additionally advantageous in that the produced tissue fits precisely into the defect to be provided for, and so an optimum functional and aesthetic result is obtained.

These bioreactors can be produced in a known fashion from three-dimensional image data records of the defect to be provided for by using CAD/CAM techniques or by shaping individual, dimensionally accurate defect models produced by means of CAD/CAM techniques. The solution according the invention also comprises individual, dimensionally accurate enveloping (e.g. by coating, deep drawing) of a scaffold (that has likewise been produced in a dimensionally accurate fashion e.g. via imaging and CAD/CAM) in an individual shape such that the scaffold is tightly surrounded by the envelope, i.e. the chamber wall, and perfused by the perfusion medium. Connections and inlets are worked into this envelope; line systems can in this case be worked into the scaffold.

A particular advantage of the bioreactor according to the invention is that single-use bioreactors can be produced, which are individually tailored to the desired shape of the tissue to be implanted, i.e. the tissue to be produced.

Dependent claims 2 to 14 reproduce further advantageous refinements of the invention according to claim 1, without restricting the latter.

The tissue growing in the bioreactor is supplied via an attached, self-regulating perfusion system that preferably acts in a pulsatile fashion, by means of which an adapted nutrient medium (“perfusion medium”) is transported. The arrows on inlet 2 and outlet 3 in FIGS. 1A to 1C reproduce the direction of the medium perfusion.

The perfusion medium is pumped into the resorbable or non-resorbable hollow line system integrated into the chamber, blood vessels or blood-vessel equivalents produced by means of tissue engineering, or the porous scaffold and thus distributed whilst taking into account the shape of the tissue structure to be cultivated. After the perfusion medium has flown through the scaffold or the hollow line system and supplied oxygen and nutrients to the tissue situated in the chamber, it flows out of the interior (cavity) of the bioreactor through the outlet.

The resorbable or permanent hollow line system integrated into the bioreactor, or the distribution via the porous scaffold, initially supplies the tissue, possibly until the latter can supply itself as a result of an individual vessel system forming or until it is implanted. The growth of a vessel system may be promoted by the hydrodynamic load that is exerted on the direct vicinity of the hollow line system due to the pulsatile perfusion. The transmission of the mechanical impulses and the intensity thereof can be varied depending on the flexibility of the selected material composition of the hollow line system. An artificial vessel system can be produced by means of a predefined two-dimensional matrix or a three-dimensional mesh.

As an alternative to an artificial hollow line system, i.e. a hollow line system produced by scaffold materials, or as an alternative to simply a porous, perfusable scaffold, a blood-vessel system, which is produced without a scaffold by means of tissue engineering, and the developing vessel sproutings thereof, or a combination of synthetically resorbable scaffolds and vessels produced by means of tissue engineering, can also distribute the perfusion medium and hence supply the surrounding tissue.

Furthermore, devices for monitoring (observing and controlling) can be integrated into the elastic wall of the cavity of the bioreactor. These include, for example, viewing panes for direct optical observation (e.g. by microscopy, fluorescence microscopy, laser scanning microscopy, etc.). Functional monitoring is brought about by means of a probe system, which monitors matter concentrations and physical or chemical variables such as e.g. O₂ and CO₂ concentration, pressure in the chamber and in the remaining bioreactor, partial pressure of oxygen, pH, flow velocity and temperature. The stretch of the elastic walls can be monitored by strain gauges. Monitoring moreover actively contributes to regulating the growth conditions in the bioreactor system because it is included in a closed-loop control as a sensor system.

A particular advantage of this procedure is the ability to produce individually disposable bioreactors that are tailored to the desired shape of the tissue to be implanted, i.e. the tissue to be produced.

All advantages of the invention just listed above thus clearly contribute to improving previous bioreactor systems, the growth conditions in bioreactors and the quality of cultivated tissue structures, and have positive effects on tissue engineering (cell and tissue cultivation) in general and in particular.

The invention will be explained in more detail in the following text with the aid of exemplary embodiments, without this having exhaustively illustrated all options for using the invention.

### EXAMPLE 1

### Producing an Individual, Elastic Pre-Shaped Bioreactor

Following a three-dimensional representation of a human or animal tissue defect by means of known imaging methods, a three-dimensional data record is calculated for planning the shape of the bioreactor so as to be able to obtain the shape of the tissue to be produced according to the invention. Herein, this raw data used for this purpose can originate from various known imaging modalities (CT, MRI (magnetic resonance imaging), ultrasound, etc.) and is preprocessed by suitable image processing methods.

The three-dimensional wireframe model finally exported to a CAD/CAM system can be translated with high precision into a 3D model of the bioreactor by a 3D printer, a CNC mill or any other instrument for three-dimensional shaping.

In the process, the bioreactor is directly produced from elastic biocompatible material (elastomers, e.g. silicones) or the 3D model is used as a mold for the cast.

Proceeding from three-dimensional patient data (CT, MRI, further modalities), a three-dimensional wireframe model with an appropriate spatial resolution in the respective shape of the required tissue is produced by means of a CAD/CAM system after appropriate preprocessing of the raw data. The geometry data of the wireframe model is subsequently loaded into an adequate system for three-dimensional shaping (3D printer, CNC mill, etc.) and the bioreactor is thus produced with very high precision.

Either the bioreactor is produced directly or the mold for the cast of the bioreactor is first of all produced using an appropriate material. Both single use and multi-use, i.e. reusable, bioreactors can be produced. FIGS. 1A to 1C show variants of the elastic bioreactor for producing tissues with an elastic envelope 1 in the shape of a defect.

A further alternative option consists of dimensionally accurately enveloping 1 scaffolds, for example by deep drawing or coating with suitable plastics. In the process, the shape is prescribed by the scaffold 4, i.e. the latter has been produced according to the image data of the defect by means of CAD/CAM where necessary. Connections 2 and inlets 3 are worked into this envelope 1; line systems can in this case be worked into the scaffold. After enveloping and attaching the connections, the bioreactor including the enveloped scaffold is ready for a single use. The arrows in FIGS. 1A to 1C at the inlet opening 2 and the outlet opening 3 show the direction of the perfusion of the medium.

Corresponding connections for monitoring and perfusion systems, and also force transmission points for mechanical loads are already taken into account during the planning phase of the bioreactor, and these are worked into the three-dimensional wireframe model in the CAD/CAM system.

Line systems, hollow fiber systems or negative shapes for line systems, which, after removal, leave channels through which a medium can flow, can be worked into the cavity or the walls in the same fashion.

### EXAMPLE 2

### Implementing Supplying Vessels (FIGS. 1A to 1C)

There are options for installing a supplying vessel or line system into the tissue as a result of installing pre-shaped hollow fiber or line systems, as a result of tissue engineering vessels, or as a result of a combination of the two. Thus, for example, it would be possible for a line system to be produced by a casting method. Wires 6 made of a suitable smooth material are laid in the bioreactor and connect the inlet opening 2 with the outlet opening 3. The bioreactor is filled with particles of a carrier material, which was populated by the cells of the target tissue (covered microcarrier). These were initially cultivated separately until the cells (stem cells, pre-differentiated cells, or differentiated cells) reached a certain density. They are then placed into the bioreactor together with fibrin, the latter polymerizing as a result of adding thrombin, and so the covered microcarrier/particles of a carrier material may be available in a fibrin matrix. If need be, endothelial cells can also be added such that a capillary-like system can be formed. The wires 6 are removed, and lines, tubes or channels remain between the inlet 2 and the outlet 3, through which the medium can perfuse. (FIG. 1A shows a bioreactor for producing tissue, with removable wires as placeholders for channels and lines).

If necessary, the channels can additionally be populated in sequence by vessel wall cells (smooth muscle cells, endothelial cells). The growth of a vessel system may be promoted by the hydrodynamic load exerted on the direct vicinity of the artificial vessel walls due to the pulsatile perfusion. The perfusion medium is distributed, and hence surrounding tissue is supplied, by this artificial blood-vessel system, produced by means of tissue engineering, and by the vessel sproutings developing during the cultivation period.

Alternatively, a pre-shaped, possibly resorbable, hollow fiber system 5 can also be laid, which is then used as tube system for the supply (FIG. 1B, elastic bioreactor for producing tissue using a hollow fiber system). The tissue is at first supplied by the resorbable or permanent hollow line system 5 integrated into the bioreactor, or by the distribution via the porous scaffold, if necessary until said tissue can supply itself as a result of forming its own vessel system or until said tissue is implanted. The initial supplier is resorbed at a later stage and replaced by vessels, or resorbed without a function if the perfusion via a collateral blood supply suffices after the transplantation.

Furthermore, use can merely be made of a porous scaffold 4, with the nutrient medium flowing through the pores of said scaffold. FIG. 1C shows an elastic bioreactor for producing tissue using a porous scaffold for distributing the medium. If need be, channels and lines 7 (with larger pore and channel diameters), possibly made of resorbable material, can be worked in such that a through-flow is maintained even in the case where the cells proliferate, and the vessel wall cells can be populated such that vessels can form.

The arrows visible in the interior in FIG. 1C show the flow direction of the medium in the porous scaffold.

### EXAMPLE 3

### Bioreactor for Replacing Soft Tissue (FIG. 1C)

A virtual 3D model of the soft-tissue defect to be supplied is generated, and this model is used as a basis of a dimensionally accurate scaffold 4 produced (remaining soft) by means of CAD/CAM techniques. The scaffold is porous and contains channels 7 for the perfusion, which channels open up for the inlet 2 and outlet 3 at the pre-calculated sites. The porosity of the scaffold ensures that the medium can distribute sufficiently well in the entire scaffold from the lines. (The arrows visible in the interior in FIG. 1C show the flow direction of the medium in the porous scaffold.)

The scaffold is then covered in a film-like manner by an elastic plastic, e.g. by deep drawing or coating (preferably silicones). Prefabricated connectors are polymerized into the predefined inlets and entry points of probes. Hence, an individual reactor is created for an individual defect. Populating can then be brought about by inoculation with suspended cells (a number of times where necessary), sequentially if need be (first mesenchymal cells of the mesenchyme, then vessel wall and endothelial cells for the vessels).

In principle, the method can be applied to any vascularized tissue.

### EXAMPLE 4

### Additionally Integrating Devices into the Elastic Envelope (Chamber Wall) of the Bioreactor

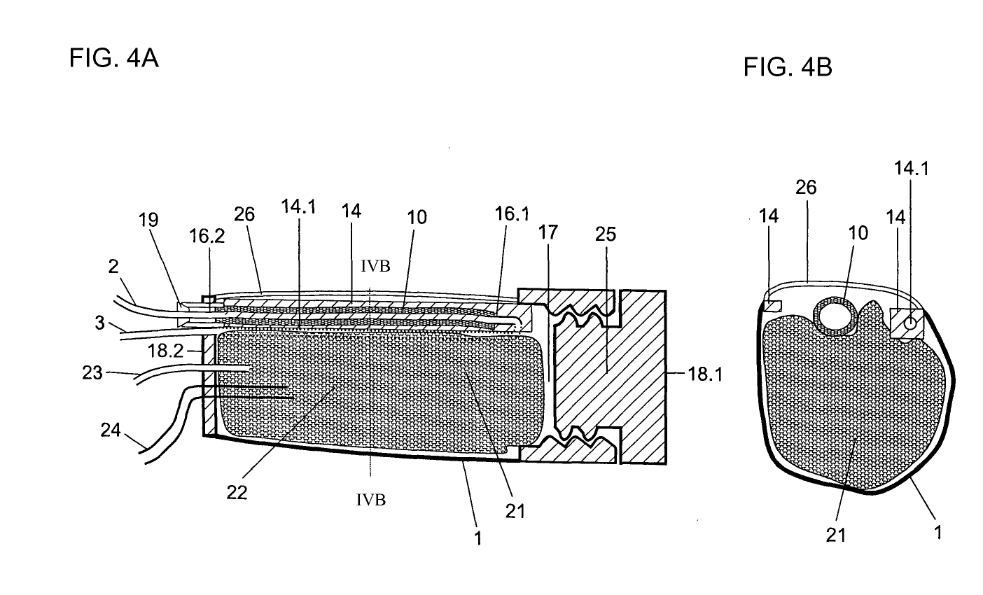

The material used in producing the bioreactor has an effect on the transparency of the chamber wall. Hence, it may be necessary, particularly in the case of opaque or insufficiently transparent materials, to integrate viewing panes for optical monitoring (observing and controlling) into the wall (see the example of a transparent film as monitoring window 26 in the experimental bioreactor according to FIGS. 4a and 4b). Furthermore, additional devices can be integrated into the wall for regulating the local or global resilience of the bioreactor system on the basis of e.g. highly elastic, biocompatible membranes.

Monitoring (observing and controlling) the growth parameters in the interior of the bioreactor can be brought about by an appropriate probe system, which is installed via predefined connections in the chamber. In the process, matter concentrations are measured, as are physical or chemical variables such as e.g. O₂ and CO₂ concentration, pressure, partial pressure of oxygen, pH, viscosity, flow velocity and temperature. Monitoring moreover actively contributes to regulating the growth conditions in the bioreactor system because it is included in the closed-loop control as a sensor system.

### EXAMPLE 5

### Producing a Blood-Vessel System by Means of Tissue Engineering

The perfusion medium is distributed, and hence the surrounding tissue is supplied, by an artificial, supplying blood-vessel system, produced by means of tissue engineering, and the vessel sproutings of the system developing during the cultivation period.

### EXAMPLE 6

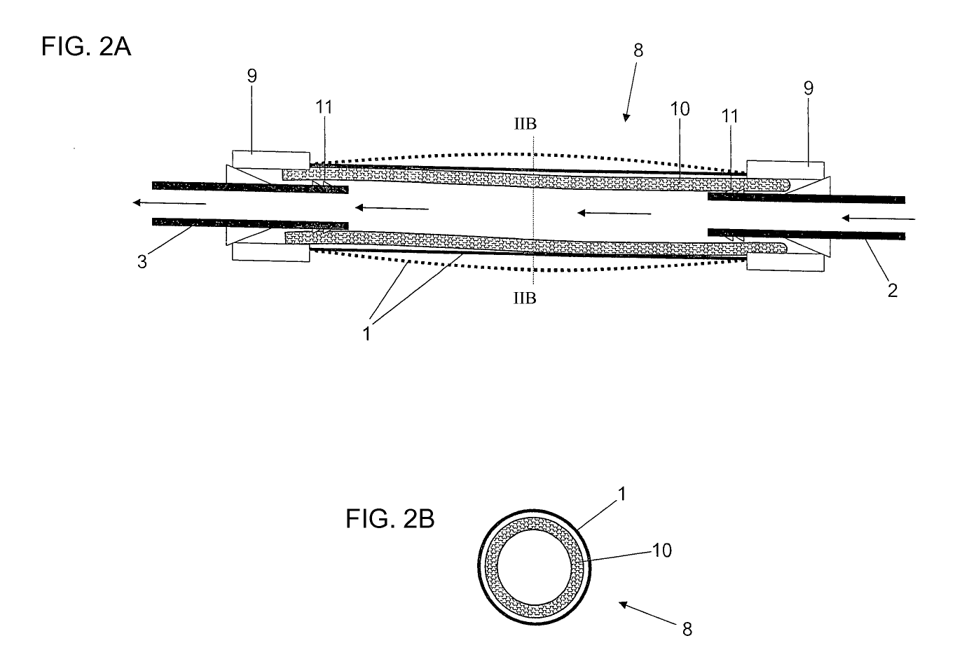

### Producing Blood Vessels or Blood-Vessel Networks, Other Tissues (FIGS. 2a and 2b, and Also 3a and 3b)

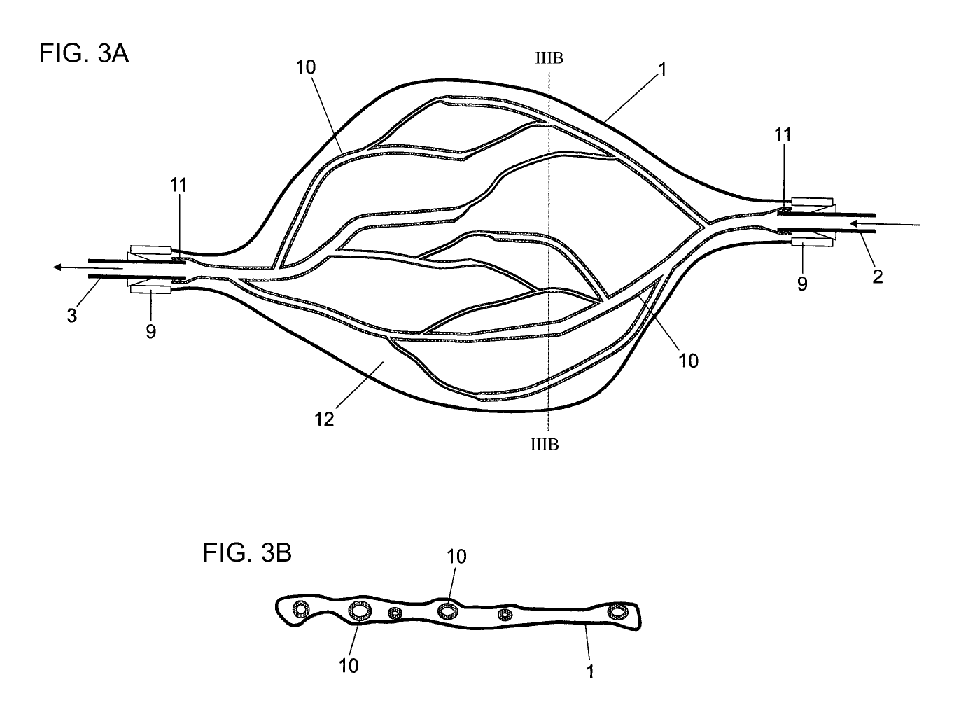

The simplest geometry is available when producing a single blood vessel. In this case, the bioreactor for producing a blood vessel according to FIGS. 2a and 2b merely consists of a cylindrical elastic body 8, which corresponds to the external diameter of the blood vessel. At the ends (FIG. 2), there respectively are couplings/connections 9 to which the vessel/vessel equivalent 10 (e.g. an elastic, resorbable scaffold material with a tubular shape) can be clamped (FIG. 2a). In the process, the latter is pushed onto tube clips 11, and these are in turn placed into a Luer-Lock fitting on the connection 9 of the bioreactor, which brings about the seal (on both sides). The structure can then be perfused by the medium and populated by cells if this has not already happened prior to clamping (smooth muscle cells and/or progenitor cells and/or endothelial cells, sequentially where necessary). The arrows in FIG. 2a and FIG. 3a correspond to the direction of the flow of the medium.

Perfusion takes place using a pulsatile perfusion mode where possible, which simulates the blood-pressure conditions in natural vessels or slowly increases said blood-pressure conditions from low pressures to physiological pressures. As a result, this eventually forms a natural, resistive (against pressure) vessel wall with physiological compliance, etc. (Two dotted lines are drawn in FIG. 2a, which illustrate (in a superposed fashion) the elastic envelope 1 in the case of deflection by perfusion pressure.)

A blood-vessel network is produced in a similar fashion, except for that a more complicated geometry of a branched network 12 in accordance with FIG. 3 is used instead of the tubular structure and bioreactor. (FIG. 3a shows a plan view and FIG. 3b shows a cross section of the elastic bioreactor for producing a blood-vessel network or a blood-vessel-network equivalent). The basic procedure is identical.

### EXAMPLE 7

### Producing a Vascularized Tissue with a Compartmented Bioreactor

In some applications, it is expedient not to produce the entire structure in one step. This is afforded by the compartmented version of the bioreactor. By way of example, in order to produce a vascularized soft tissue, the blood-vessel network, as described in example 6, is first of all produced in one compartment and then a separation wall is opened to the second sterile, still unused compartment. The actual transplant tissue or tissue equivalent is then deposited therein (as a scaffold with cells, a scaffold or particles that can be populated, or cells without a scaffold), and so it is fed via the already present vessel network.

### EXAMPLE 8

### Connecting and Operating the Self-Regulating Pulsatile Perfusion System

A self-regulating pulsatile perfusion system is connected to the bioreactor or the hollow line system established thereby and is used for simulating physiological or experimental pressure conditions.

### EXAMPLE 9

### Applications

Application options for the bioreactor according to the invention present themselves whereever the interactions between vessel and stroma and mesenchymal tissue or other tissues play a role. These are many fields in addition to the previously sketched out applications in regenerative medicine and tissue engineering. As sketched out in the preceding examples, the system can likewise be operated with natural, explanted tissues and vessels, analogously to the tissue equivalents or artificial tissues. This results in a broad field of application. Said applications include, for example, basic-research oriented examinations, in particular in the research of circulatory diseases, and also many metabolic disorders, such as e.g. obesity, in which the interaction between vessels and fat cells plays an important role. Furthermore, it can be useful as a metastasis model in oncology research. Questions in respect of wound healing can be answered therewith, and it can also be used as an angiogenesis model in basic research. A substantial branch is also the application thereof in testing pharmacological agents, e.g. testing the transfer of pharmacological agents into the interstitium or other questions. Here, and in other applications, it can also be used as a replacement for animal testing.

### EXAMPLE 10

### Variant for Experimental Purposes (FIGS. 4a and 4b)

A miniaturized embodiment variant for experimental applications is described in the following text (FIGS. 4a and 4b), in which a vessel equivalent or a blood vessel 10 is cultivated together with a tissue section (structure/tissue piece) 21 such that it can be subjected to comprehensive monitoring (observation and control). Said embodiment consists of a thin support 14 being integrated into the chamber wall and connecting both end faces, at the ends of which (the support) connections 16.1 and 16.2 are respectively attached that serve for connecting a blood vessel or a blood-vessel equivalent. The connection 16.2 at the inlet 2 has to be designed such that the vessel/vessel equivalent 10 can be introduced in a sterile fashion through the large opening for loading and construction 17, comprising a screw top 25, and can be coupled to the end face 18.2. For this purpose, the end face 18.2 has been provided with a smaller opening with a flange, through which a coupling 19 with a tube clip can be inserted from the outside, and to which the vessel/vessel equivalent 10 is fixed. This coupling 19 is attached in a liquid-sealed fashion to the flange, e.g. using the Luer-Lock principle, and fixes the vessel/vessel equivalent. The vessel/vessel equivalent is subsequently attached to the connection 16.1 (e.g. tube clip). The line for the outlet 3 is guided in the support 8 or along the latter (14.1). Hence a tubular structure is perfused, whilst all other features of the chamber are maintained. This affords the possibility of producing or simulating a blood vessel by means of tissue engineering, which blood vessel is in direct contact with a supplied tissue section (structure/tissue piece) 21. By way of example, this affords the possibility of examining conditions in which sproutings (small blood vessels) grow into the attached tissue from the central vessel. If human or animal blood vessels or tissue are taken instead of structures produced by means of tissue engineering, it is also possible to examine physiological or pathological processes in vitro that until now were reserved for animal testing. This preferably holds true for pathological and physiological processes in vessels or the circulatory system, for obesity research and for testing pharmacological substances in which the interactions between blood vessels and tissue play a role.

1-44. (canceled)

45. A perfusable bioreactor for producing a tissue selected from the group consisting of human tissues, animal tissues and tissue equivalents, the perfusable bioreactor comprising:
an envelope defining and enclosing an internal space, said envelope having at least one inlet and at least one outlet for a liquid nutrient medium, said envelope being connectable to a unit for generating a perfusion pressure of the liquid nutrient medium, said envelope having at least one subsegment formed of an elastic material; and
a structure disposed in said internal space for cultivating the tissue.

46. The bioreactor according to claim 45, wherein said envelope has an envelope surface with an inner surface, said subsegment contains more than 50% of said inner surface of said envelope surface.

47. The bioreactor according to claim 45, wherein said structure is selected from the group consisting of perfusable resorbable porous scaffolds, non-resorbable porous scaffolds, line systems, hollow fiber systems, and a combination of at least two of the structures for a distributing a culture medium in the tissue to be produced.

48. The bioreactor according to claim 45, wherein the liquid nutrient medium can flow through said structure disposed in said internal space.

49. The bioreactor according to claim 45, wherein said internal space has an internal contour which prescribes at least 50% of an external contour of the tissue.

50. The bioreactor according to claim 45, wherein said structure is a scaffold filling said internal space with a congruent shape.

51. The bioreactor according to claim 45, wherein the bioreactor is operated as a unit in conjunction with a self-regulating pulsating perfusion system.

52. The bioreactor according to claim 45, wherein a physical pressure-load regime that can be generated in said internal space corresponds to a physical pressure-load regime that is exerted on the tissue under normal physiological or pathological conditions in a living human organism or living animal organism.

53. The bioreactor according to claim 45, wherein an elasticity of said elastic material of said subsegment can be set such that a stretch of the tissue together with an extensibility of said envelope as a result of the perfusion pressure generated in said internal space corresponds to physiological values or pathological values of a tissue compliance of the tissue to be produced.

54. The bioreactor according to claim 45, wherein connections and devices for monitoring have already been taken into account and worked in at a start of a production process, including when creating a model using a CAD/CAM system.

55. The bioreactor according to claim 45, wherein said internal space is subdivided into at least two compartments, which have individual perfusion circuits and are subdivided by walls selected from the group consisting of removable separation walls and non-removable separation walls.

56. The bioreactor according to claim 45, further comprising a viewing pane directly inserted into said envelope for optical or functional monitoring.

57. The bioreactor according to claim 45, wherein the bioreactor is a disposable reactor and can be used for a single use.

58. The bioreactor according to claim 45, wherein said structure is a scaffold filling said internal space and an internal contour of the bioreactor is produced by enveloping said scaffold by said envelope, wherein said scaffold is formed according to a defect to be provided for.

59. The bioreactor according to claim 45, wherein said envelope has an envelope surface with an inner surface, said subsegment contains more than 75% of said inner surface of said envelope surface.

60. The bioreactor according to claim 45, wherein said internal space has an internal contour which prescribes at least 70% of an external contour of the tissue.

61. The bioreactor according to claim 45, wherein said internal space has an internal contour which prescribes 100% of an external contour of the tissue.

62. A tissue producing method, which comprises the step of:
providing a perfusable bioreactor containing:
an envelope defining and enclosing an internal space, the envelope having at least one inlet and at least one outlet for a liquid nutrient medium, the envelope being connectable to a unit for generating a perfusion pressure of the liquid nutrient medium, the envelope having at least one subsegment formed of an elastic material;
a structure disposed in the internal space for cultivating tissues; and
producing a tissue selected from the group consisting of human tissues, animal tissues and tissue equivalents in the perfusable bioreactor.

63. The method according to claim 62, which further comprises producing the tissue equivalent by filling a cavity in the internal space with a particulate carrier material that has been prepopulated with mesenchymal cells.

64. The method according to claim 63, which further comprises adding endothelial cells in addition to the mesenchymal cells.

65. The method according to claim 64, which further comprises adding the particulate carrier material or the endothelial cells in a fibrin matrix.

66. The method according to claim 62, which further comprises introducing the structure into the internal space of the perfusable bioreactor and a remaining cavity in the internal space is filled by the liquid nutrient medium and perfused by the liquid nutrient medium via the inlet and the outlet.

67. The method according to claim 62, which further comprises filling a cavity in the internal space with a particulate carrier material that has been prepopulated with mesenchymal cells.

68. The method according to claim 67, which further comprise adding endothelial cells in addition to the mesenchymal cells.

69. The method according to claim 68, which further comprises adding the particulate carrier material or the endothelial cells in a fibrin matrix.

70. The method according to claim 62, which further comprises brining about production by means of the structure being scaffold, which is surrounded by the envelope in a dimensionally accurate fashion and through which perfusion can take place as a result of a porosity of the scaffold or line structures of the scaffold.

71. The method according to claim 62, which further comprises:
exerting a physical pressure-load regime onto the structure corresponding to a physical pressure-load regime that is exerted on a produced tissue under normal living conditions in a living human organism or a living animal organism; and
introducing the liquid nutrient medium into the internal space via the inlet with a positive pressure and in a pulsating fashion, and leaves the internal space at least via the at least one outlet.

72. The method according to claim 62, which further comprises filling a cavity in the internal space with a particulate, resorbable, carrier material that has been prepopulated with mesenchymal cells.

73. The method according to claim 62, which further comprises producing the tissue equivalent by filling a cavity in the internal space with a particulate, resorbable, carrier material that has been prepopulated with mesenchymal cells.

74. A method for producing an artificial, supplying blood-vessel system produced by means of tissue engineering, the blood-vessel system develops vessel sproutings during a cultivation period and thereby assumes a role of distributing a perfusion medium or supplying a surrounding tissue, which comprises the steps of:
providing a perfusable bioreactor containing:
an envelope defining and enclosing an internal space, the envelope having at least one inlet and at least one outlet for a liquid nutrient medium, the envelope being connectable to a unit for generating a perfusion pressure of the liquid nutrient medium, the envelope having at least one subsegment formed of an elastic material; and
a structure disposed in the internal space for cultivating the blood-vessel system.

75. A method for producing a perfusable bioreactor for producing a tissue selected from the group consisting of human tissues, animal tissues and tissue equivalents, which comprises the steps of:
providing an envelope defining and enclosing an internal space, the envelope having at least one inlet and at least one outlet for a liquid nutrient medium, the envelope being connectable to a unit for generating a perfusion pressure of the liquid nutrient medium, the envelope having at least one subsegment formed of an elastic material; and
providing a structure disposed in the internal space for cultivating the tissue.

76. The method according to claim 75, which further comprises producing the perfusable bioreactor by means of CAD/CAM technologies according to imaging data of a given defect to be provided for.

77. The method according to claim 76, which further comprises taking account of connections, couplings and interfaces for guiding the liquid nutrient medium or the guiding of the liquid nutrient medium and monitoring are worked in at a beginning of a production process, namely when creating a model with the CAD/CAM system.

78. The method according to claim 75, which further comprises bringing about production at least in part by covering, namely by deep drawing or coating an individual scaffold.

79. The method according to claim 75, which further comprises selecting the structure from the group consisting of hollow fiber systems, line systems and scaffold structures.

80. A method for producing a resorbable, temporary hollow fiber system made of a mixture of various resorbable, biocompatible materials for supplying a cultivated tissue with oxygen and nutrients in a bioreactor, which comprises the step of:
providing a perfusable bioreactor containing an envelope defining and enclosing an internal space, the envelope having at least one inlet and at least one outlet for a liquid nutrient medium, the envelope being connectable to a unit for generating a perfusion pressure of the liquid nutrient medium, the envelope having at least one subsegment formed of an elastic material; and
disposing a structure in the internal space for cultivating the resorbable, temporary hollow fiber system in the perfusable bioreactor.

81. A method for producing a resorbable, temporary hollow fiber system, which comprises the steps of:
providing a perfusable bioreactor containing an envelope defining and enclosing an internal space, the envelope having at least one inlet and at least one outlet for a liquid nutrient medium, the envelope being connectable to a unit for generating a perfusion pressure of the liquid nutrient medium, the envelope having at least one subsegment formed of an elastic material;
disposing a structure in the internal space for cultivating the resorbable, temporary hollow fiber system in the perfusable bioreactor; and
matching the resorbable, temporary hollow fiber system in space to the shape of the perfusable bioreactor, and hence to a tissue to be produced;
connecting the perfusable bioreactor to a perfusion system.

82. A production method, which comprises the step of:
providing a perfusable bioreactor containing:
an envelope defining and enclosing an internal space, the envelope having at least one inlet and at least one outlet for a liquid nutrient medium, the envelope being connectable to a unit for generating a perfusion pressure of the liquid nutrient medium, the envelope having at least one subsegment formed of an elastic material; and
a structure disposed in the internal space for cultivating a tissue.

83. The method according to claim 82, which further comprises producing human tissue, animal tissue or scaffold-cell combinations for purposes of therapy in the perfusable bioreactor.

84. The method according to claim 82, which further comprises experimentally producing human tissue, animal tissue or scaffold-cell combinations in the perfusable bioreactor.

85. The method according to claim 82, which further comprises producing vascularized tissues in the perfusable bioreactor.

86. The method according to claim 82, which further comprises producing soft tissue or soft-tissue equivalents in the perfusable bioreactor.

87. The method according to claim 82, which further comprises producing blood vessels or blood-vessel equivalents in the perfusable bioreactor.

88. The method according to claim 82, which further comprises producing blood-vessel networks or blood-vessel-network equivalents in the perfusable bioreactor.

89. The method according to claim 82, which further comprises producing hard tissue or hard-tissue equivalents in the perfusable bioreactor.

90. The method according to claim 82, which further comprises producing combined tissues made of soft tissue, hard tissue and/or blood vessels in the perfusable bioreactor.

91. The method according to claim 82, which further comprises testing pharmacological substances in the perfusable bioreactor.

92. The method according to claim 82, which further comprises testing pharmacological substances in a field of circulatory system and obesity research in the perfusable bioreactor.

93. The method according to claim 82, which further comprises using the perfusable bioreactor for oncologic questions, including relating to metastasis or for testing of pharmacological substances.

94. The method according to claim 82, which further comprises using the perfusable bioreactor for replacing animal testing.
